(19) United States
(12) Patent Application Publication
(10) Pub. No.: US 2026/0053886 A1
(43) Pub. Date: Feb. 26, 2026

(54) ANTICANCER AGENT

(71) Applicant: The Trustees of Princeton University, Princeton, NJ (US)

(72) Inventors: Mohammad R. SEYEDSAYAMDOST, Princeton, NJ (US); Yuchen LI, Princeton, NJ (US); Seungrak LEE, Princeton, NJ (US); Esther J. HAN, New Haven, CT (US)

(73) Assignee: The Trustees of Princeton University, Princeton, NJ (US)

(21) Appl. No.: 19/100,322

(22) PCT Filed: Jun. 5, 2023
(86) PCT No.: PCT/US2023/024441

§ 371 Date: Jan. 31, 2025

Related U.S. Application Data
(60) Provisional application No. 63/395,575, filed on Aug. 5, 2022.

(51) Int. Cl. A61K 38/12; C07K 7/52

(52) U.S. Cl. CPC A61K 38/12; C07K 7/52

(57) ABSTRACT

Disclosed is a composition for use in treating colorectal cancer and/or adenocarcinoma. The compound may include momomycin or a derivative thereof in a pharmaceutically acceptable carrier, at a total concentration of at least 1 μM, such as 1-2 μM. The momomycin may be produced by growing a culture of a strain of Streptomyces rimosus, such as S. rimosus Δrim::apr^{R}, in the presence of phytosphingosine. The supernatant may be filtered and purified.

### CROSS-REFERENCE TO RELATED APPLICATIONS

The present application claims priority to U.S. provisional patent application 63/395,575, filed Aug. 5, 2022, the contents of which are hereby incorporated by reference in its entirety.

### STATEMENT REGARDING FEDERALLY SPONSORED RESEARCH OR DEVELOPMENT

This invention was made with government support under Grant No. GM140034 awarded by the National Institutes of Health. The government has certain rights in the invention.

### TECHNICAL FIELD

The present application is drawn to anticancer agents, and techniques for manufacturing such anticancer agents.

### BACKGROUND

According to the World Health Organization (WHO), cancer is among the four major non-communicable deadly diseases worldwide in from 2000-2019. While the number of cancer treatments is increasing annually, most such treatments are variations of existing treatments, using known mechanisms of action. To aid in combatting cancer, treatments utilizing heretofore unknown mechanisms of action are needed.

### BRIEF SUMMARY

In various aspects, a pharmaceutical composition, such as one for use in treating colorectal cancer and/or adenocarcinoma, may be provided. The composition may include momomycin or a derivative thereof in a pharmaceutically acceptable carrier, at a total concentration of at least 1 μM, such as 1-2 μM.

A method of treatment of colorectal cancer and/or adenocarcinoma may be provided. The method may include administering a pharmaceutical composition as disclosed herein to a patient.

A method for production of the anticancer agent momomycin or a derivative thereof may be provided. The method may include growing a culture of a strain of Streptomyces rimosus in the presence of phytosphingosine. The strain may be, e.g., S. rimosus Δrim::apr^{R}. The method may include filtering supernatant from the culture. The method may include purifying the filtered supernatant.

### BRIEF DESCRIPTION OF THE DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a chemical structure representative of the anticancer agent momomycin.

FIGS. 2A and 2B are graphs showing dose-response curves against colorectal carcinoma HCT116 (2A) and adenocarcinoma MDA-MB-231 (2B) yielding IC₅₀ values of 1.5 μg/mL (1.1 μM) and 1.7 μg/mL (1.2 μM), respectively.

FIG. 3 is an illustration of the rim biosynthetic gene cluster (BGC) in S. rimosus.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

In various aspects, a pharmaceutical composition, such as one for use in treating colorectal cancer and/or adenocarcinoma, may be provided. The composition may include momomycin or a derivative thereof in a pharmaceutically acceptable carrier, at a total concentration of at least 1 μM. In some embodiments, the concentration may be 1-2 μM. In some embodiments, the concentration may be no more than 3 μM.

As used herein, the term “derivative” has a structure derived from the structure of a parent compound (e.g., a compound disclosed herein), the structure of which is disclosed herein. Sufficiently similar to, and based on its similarity, exhibiting the same or similar activity and usefulness as the claimed compound, or as a precursor, the same or similar activity and usefulness as the claimed compound. Exemplary derivatives include salts of parent compounds, esters, amides, salts of esters or amides, and N-oxides.

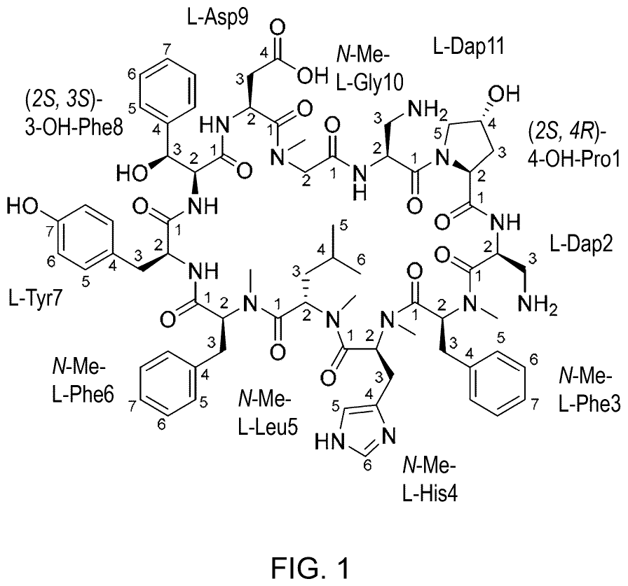

Momomycin is a small organic molecule, the structure of which can be seen in FIG. 1. It is an unusually large, 11-residue macrocyclic non-ribosomal peptide (NRP). Momomycin is a peptidic molecule, and as such, it will be understood that derivatives may be easily generated and tested broadly to optimize a particular desired pharmaceutical property. Momomycin contains three unusual amino acids (2S,4R)-4-OH-Pro, L-Dap, and (2S,3S)-3-OH-Phe. In some embodiments, a derivative may be free of an alteration of these three amino acids.

The term “peptidic molecule” (also referred to herein as “peptide molecule”), as used herein, refers to any naturally occurring or synthetic (e.g., generated by chemical synthesis or recombinant DNA technology) macromolecule, which may be linear macromolecules, comprising a plurality of natural or modified amino acid residues connected via peptide bonds. Such peptides may form oligomers consisting of at least two identical or different peptide molecules.

In various aspects, momomycin may be utilized as part of an antibody-drug conjugate (ADC). ADCs are known in the art—for example, the FDA has approved adotrastuzumab emtansine for use in treating HER2-positive, late-stage (metastatic) breast cancer.

Many ADCs have a general formula AB-(L-ACA)ₓ, where x is an integer from 1-8. In some embodiments, x is 1. In some embodiments, x>1. AB is an appropriate antibody for targeting the cancer in question (such will be known to those of skill in the art). ACA is the anticancer agent (here, momomycin or a derivative thereof). L is a linker, such as a peptidomimetic linker, for connecting the antibody and the anti-cancer agent. The linker may be a non-cleavable linker. Momomycin and its derivatives have several moieties or anchor points (including, e.g., primary sidechain amines and sidechain carboxylic acids) that a linker can use to couple the momomycin to an appropriate antibody.

The term “linker” refers to a chemical moiety or bond that covalently attaches two or more molecules, such as a targeting moiety and a drug.

The term “non-cleavable linker” refers to a stable linker that has the property of being more stable in vivo than either the therapeutic or the targeting moiety under the same physiological conditions. Examples of non-cleavable linkers include linkers that contain polyethylene glycol chains or polyethylene chains that are not acid or base sensitive (such as hydrazone containing linkers), are not sensitive to reducing or oxidizing agents (such as those containing disulfide linkages), and are not sensitive to enzymes that may be found in cells or circulatory system. Non-limiting examples of cleavable linkers include linkers that contain non-hindered glutathione sensitive disulfides, esters, peptide sequences sensitive to the peptidases such as cathepsin or plasmin, or pH sensitive hydrazones.

The linker may follow a formula CAM-NPM-SP, where CAM material that may be covalently attached to the antibody, NPM is a non-peptide moiety known to those of skill in the art, and SP is a spacer that may be attached to the anticancer agent.

The effect of momomycin on microbial growth was tested. No noteworthy activity was observed in assays against a number of common bacterial pathogens, human microbiome commensal strains, and fungal model organisms, including E. coli K12, P. aeruginosa PAO1, V. cholerae, B. subtilis 168, B. subtilis 3610, S. aureus Newman, E. faecalis OG1RF, S. epidermidis, M. luteus, S. cerevisiae, and S. pombe. All had a minimum inhibitory concentration (MIC) above 64 μg/mL, except for M. luteus, whose MIC was 64 μg/mL.

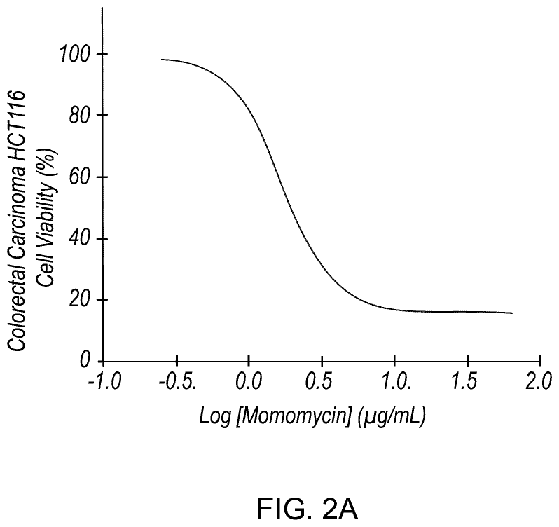

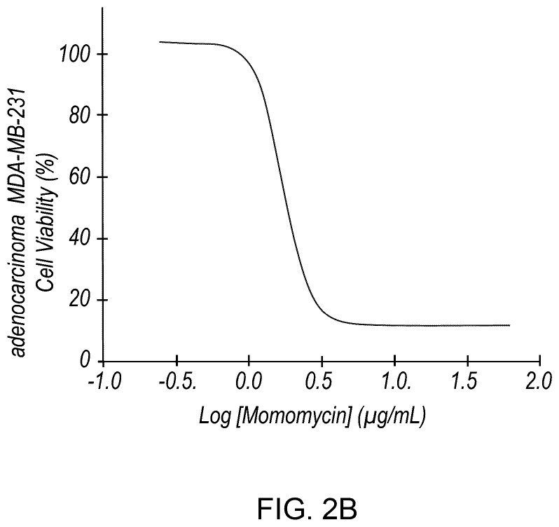

When tested in anticancer assays, however, momomycin exhibited strong growth inhibition against colon cancer cell line HCT116 and breast cancer cell line MDA-MB-231 with half-maximal inhibitory concentrations (IC₅₀) of about 1.1 μM and 1.2 M, respectively. See FIGS. 2A and 2B.

As used herein, the term “pharmaceutically acceptable carrier” means a non-toxic, inert solid, semi-solid or liquid filler, diluent, encapsulating material or formulation auxiliary of any type. In various aspects, the pharmaceutical compositions disclosed herein may be administered to humans and other animals orally, rectally, parenterally, intraperitoneally, topically (as by powders, ointments, or drops), or as an oral or nasal spray.

When introduced orally, the composition may be formulated as a liquid. The liquid may include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (such as, e.g., corn oil, olive oil, or castor oil), glycerol, tetrahydrofurfuryl alcohol, polyethylene glycols and fatty acid esters of sorbitan, and mixtures thereof. The oral compositions may include one or more adjuvants such as wetting agents, emulsifying and suspending agents, and flavoring agents.

When introduced via an injection, the composition may be prepared as a sterile injectable aqueous or oleaginous suspension. Such compositions may be formulated according to the known art using suitable dispersing or wetting agents and suspending agents.

When using a solid dose form, the composition may include fillers in soft and hard-filled gelatin capsules using such excipients as lactose or milk sugar as well as high molecular weight polyethylene glycols and the like.

The anticancer agent may also be in micro-encapsulated form with one or more excipients as noted above. The solid dosage forms of tablets, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents.

If it is desired to extend or prolong the effect of a drug (or to delay the release of the drug), it is often desirable to slow the absorption of the drug from subcutaneous or intramuscular injection. This may be accomplished by any known technique. For example, the composition may use a liquid suspension of crystalline or amorphous material with poor water solubility. The rate of absorption of the drug then depends upon its rate of dissolution which may depend upon crystal size and crystalline form. Alternatively, this may be accomplished by dissolving or suspending the drug in an oil vehicle.

Thus, a method of treatment of colorectal cancer and/or adenocarcinoma may be provided. The method may include administering a pharmaceutical composition as disclosed herein to a patient.

A method for production of the anticancer agent momomycin or a derivative thereof may be provided. The method may include growing a culture of a strain of Streptomyces rimosus in the presence of phytosphingosine. The strain may be, e.g., S. rimosus Δrim::apr^{R}.

While phytosphingosine activated the production of this metabolite at low concentrations, it exhibited antimicrobial activity against S. rimosus Δrim::aprR at high doses with a half-maximal inhibitory concentration (IC₅₀) of 37.8 μM. The half-maximal elicitor concentration (EC₅₀) was in a similar concentration range, 16.9 μM, connecting cytotoxicity with elicitation. In some embodiments, 30 μM of phytosphingosine, close to its IC50 concentration, may be used to induce synthesis of the cryptic metabolites. In some embodiments, the concentration of phytosphingosine is no more than 90% of the IC₈₀ concentration. In some embodiments, the concentration of phytosphingosine is no more than 80% of the IC₅₀ concentration.

The method may include filtering supernatant from the culture.

The method may include purifying the filtered supernatant.

### Example 1 (Production of S. rimosus ΔRim::Apr^{R})

Deletion of the rimocidin biosynthetic gene cluster (BGC) (rim) appears to free up resources and precursors for production of other metabolites. A rimocidin gene deletion mutant may be generated by replacing a 15-kb stretch of the rim BGC with an apramycin marker (apr^{R}) (i.e., an apramycin resistance gene cassette) via insertional mutagenesis.

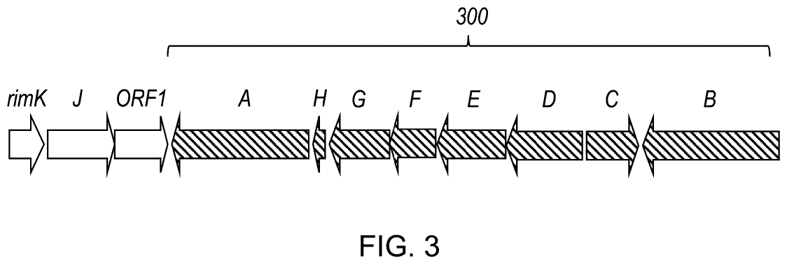

Referring to FIG. 3, a ˜15-kb section 300 of the rim BGC was selected to be deleted to generate S. rimosus Δrim::apr^{R}. In FIG. 3, “A” and “B” are Type I polyketide synthases (PKSs), “C” is Tyrosine phosphatase, “D” is Cholesterol Oxidase, “E” is Glycosyltransferase, “F” is Aminotransferase, “G” is Cytochrome P450, “H” is Ferredoxin, “J” is Crotonyl-CoA reductase, and “ORF1” is Transposase. As shown, the section to be deleted includes A, B, C, D, E, F, G, and H.

Around 2-kb fragments upstream and downstream of targeted rim genes were amplified via PCR with appropriate primers. The fragments were purified by gel extraction, ligated separately into pBluescript KS(+) (ampicillin-resistant, Apr^{R}) at EcoRV cutting site, and transformed into chemically competent E. coli DH5α cells, which were then plated on LB Amp (100 μg/mL) agar. Colonies bearing the recombinant plasmid, selected by blue/white screen method and Amp resistance were inoculated into LB Amp. Recombinant plasmids were purified from overnight culture via Qiagen Miniprep Kit and confirmed for the correct sequence. The plasmids were then digested with appropriate restriction enzymes, the recognition sequences of which were added at the end of the 2-kb fragments by primers mentioned above. The fragments were purified via gel extraction and used as homologous arms that enabled the precise insertion of the apramycin resistance gene (apr^{R}) cassette into the genome. The apr^{R} cassette was acquired by EcoRI/HindIII digestion of pIJ773 vector. The conjugation plasmid pJTU1289, which contained amp^{R} and thiostrepton resistance genes (tsr^{R}), was digested with XbaI/KpnI. Next, a four-fragment ligation was carried out at room temperature (RT) for 2 h using the cut pJTU1289 vector, apr^{R} cassette, and two homologous arms. The reaction product was transformed into chemically competent E. coli DH5α cells and plated onto LB Amp agar. Single colonies were inoculated into LB Amp, from which the conjugation plasmids were purified. To confirm the successful ligation of all desired fragments, the conjugation plasmids were cut with XbaI, EcoRI, HindIII, and KpnI. The reaction products were resolved on agarose gel and the candidate that rendered DNA bands with the correct lengths was selected.

The conjugation plasmid (Apr^{R}, Amp^{R}, Tsr^{R}) was transformed into chemically competent E. coli ET12567 (methylation defective, chloramphenicol-resistant (Cm^{R})) containing the mobilizing vector pUZ8002 (kanamycin-resistant, Kan^{R}). E. coli ET12567 carrying the desired plasmid was inoculated into LB Amp, Kan (50 μg/mL), Cm (25 μg/mL) and incubated at 37° C./200 rpm. When OD₆₀₀ reached 0.4-0.6, cells were pelleted and washed twice with LB to remove residual antibiotics, after which the wet weight of the pellet was measured in an Eppendorf tube. S. rimosus spores were grown on SFM plates and collected after 7 d into an Eppendorf tube with wet cotton swabs. The spores were washed with TES buffer (50 mM, pH 8.0), resuspended in 500 μL TES buffer, and heat-shocked at 40° C. for 10 min. Immediately after heat shock, the spores were cooled on ice for 3 min, supplemented with 500 μL spore activating solution (1% (w/v) yeast extract, 1% casamino acids, and 5 M CaCl2)), and incubated at 37° C./200 rpm for 2.5 h. The spores were then collected by centrifugation (5000 g, 5 min) and the wet weight of spores was measured.

To perform conjugation, E. coli ET12567 cells and S. rimosus spores were diluted 10-fold (w/v), and 25 μL of each were mixed and plated on SFM agar. After incubation at 30° C. for 14 h, the plate was flooded with 1 mL aqueous solution of apramycin (1 mg/mL) and trimethoprim (Tmp, 50 μg/mL). The plate was air-dried and incubated at 30° C. for 2-3 d, after which single colonies were streaked out on SFM Apr/Tmp to further validate the exconjugants. Next, single colonies were inoculated into TSBY medium without antibiotics to facilitate the process of homologous recombination. The overnight cultures grown at 30° C./250 rpm were diluted 10³-10⁴-fold and plated on SFM Apr (1 mg/mL). Single colonies were picked and tested on SFM Apr (1 mg/mL) and SFM Tsr (50 μg/mL). Those that are resistant to Apr but not Tsr were selected as successful knock-out mutants, in which double-crossover was completed between rim genes and the conjugational plasmid. In the end, genomic DNA of the mutants were extracted and the incorporation of the apr^{R} cassette was further verified via PCR using appropriate primers.

### Example 2 (Small-Scale Production of Momomycin)

A seed culture of S. rimosus Δrim::apr^{R} was inoculated in TSBY medium as described above, which was used to start fermentation cultures in 125 mL Erlenmeyer flasks with the final concentration of cells of 0.05% (w/v). Each flask contained 25 mL R4 medium, supplemented with various concentrations of elicitors or equal volumes of DMSO as control. After 5 d of cultivation at 30° C./250 rpm, the cultures were spun down and processed by solid phase extraction (SPE). Specifically, 5 mL of supernatant was filtered and loaded onto STRATA® C18-E column (Phenomenex, 55 μm, 70 Å, 50 mg), which had been activated with MeOH and equilibrated with H2O. The column was washed with 1 mL H2O and eluted by 1 mL MeOH. The samples in MeOH were dried in vacuo, resuspended in 50 μL MeCN in H2O, and subjected to HR-HPLC-MS analysis.

Separately, small-scale fermentation of S. rimosus Δrim::apr^{R} was performed as described above with the following concentrations of phytosphingosine: 0, 1.25, 2.5, 5, 10, 20, 40, 80, 160 μM. After 5 d, cells were collected by centrifugation (4000 g, 1 h) and the wet weight of pellets were measured.

Cell viability relative to DMSO-treated samples were graphed and fit to a dose-response curve in Prism 9 to obtain the IC₅₀ value. In addition, culture supernatants were processed via SPE as described above and samples were subjected to HR-HPLC-MS analysis. The extracted ion chromatograms of momomycin were integrated and the data were graphed to represent the induction effect of phytosphingosine on momomycin production at different concentrations. All data points were taken from biological duplicates.

### Example 3 (Large Scale Production of Momomycin)

Large-scale fermentation of S. rimosus Δrim::apr^{R} was carried out in a similar manner as that of small-scale cultures. Briefly, a 50 mL seed culture was inoculated from spore stock and grown overnight, which was used to start fermentation cultures (0.05% (w/v) cells) in multiple 1 L Erlenmeyer flasks, each containing 200 mL R4 medium and 30 μM phytosphingosine. The cultures were harvested after 7 d of cultivation at 30° C./250 rpm and spun down to render supernatant for further processing.

16 L of culture supernatants were filtered, loaded onto multiple prepacked STRATA® C18-E columns (Phenomenex, 55 μm, 70 Å, 10 g), and eluted in 100 mL 20%, 60%, and 100% MeOH in H2O stepwise.

Momomycin and longicatenamycin B were both present in the 100% MeOH fractions, which were pooled, dried, and subjected to SEPHADEX® LH-20 size-exclusion chromatography with MeOH as the mobile phase. Fractions were collected every 10 min (˜30 mL) and those containing target compounds, as judged by HR-HPLC-MS analysis, were pooled and dried. Subsequently, the sample was resuspended in 5 mL 50% MeCN in H2O and repeatedly injected onto a preparative LUNA® C18 column (Phenomenex, 5 μm, 21.2×250 mm) operating at a flow rate of 12 mL/min. Elution was performed first isocratically at 20% MeCN (+0.1% FA) in H₂O (+0.1% FA) for 5 min, followed by a gradient of 20-80% MeCN in H2O over 20 min, and finished by 100% MeCN over 7 min.

Momomycin and longicatenamycin B eluted in fractions collected at 14 and 15 min (12 mL per fraction), respectively, which were dried in vacuo separately. The sample containing momomycin was next resuspended in 1 mL MeOH and purified on an analytical Luna® Phenyl-hexyl column (Phenomenex, 5 μm, 4.6×150 mm) with a flow rate of 1 mL/min. The analyte was eluted isocratically with 23% MeCN (+0.1% formic acid (FA)) in H2O (+0.1% FA) for 20 min, followed by a gradient of 20-30% MeCN over 5 min and 30-100% MeCN over 1 min.

Momomycin eluted as a broad peak from 16-22 min during which the eluent was collected and then dried. Approximately 3 mg of pure material was obtained after this step.

Assessment by multi-dimensional nuclear magnetic resonance (NMR) spectroscopy showed typical features of a peptide skeleton (see Table 1, below). Detailed analysis of a variety of 1H-1H and 1H-13C spectra revealed the presence of 11 amino acids: 4-OH-Pro, two diaminopropionic acids (Dap), two N-Me-Phe, N-Me-His, N-Me-Leu, Tyr, 3-OH-Phe, Asp, and N-Me-Gly.

TABLE 1
(NMR assignments for momomycin in DMSO-d₆ from N- to C-
terminus. Underlined signals of N—Me-His are identified from
dataset obtained with methanol-d₄ as solvent. The structure
and numbering scheme for momomycin are shown in FIG.
1. Coupling constants (in Hz) are shown in parentheses.
(n.d. = not distinguishable)

| Residue | | δ_{H} (800 MHz) | δ_{C} |
|---|---|---|---|
| 4-OH-Pro (1) | 1 | — | 173.7 |
| | 2 | 3.0 brs | 58.9 |
| | 3 | 1.88 m, 1.90 m | 39.1 |
| | 4 | 4.23 m | 71.4 |
| | 5 | 2.77 d (11.5) | 54.6 |
| Dap (2) | 1 | — | 167.6 |
| | 2 | 5.01 brs | 48.7 |
| | 3 | 2.86, 3.32 overlap | 41.8 |
| | NH | 8.32 overlap | — |
| N—Me-Phe (3) | 1 | — | 167.3 |
| | 2 | 5.34 brs | 55.0 |
| | 3 | 2.48, 3.16 overlap | 34.1 |
| | 4 | — | 137.8 |
| | 5 | 7.13 d (7.5) | 128.7 |
| | 6 | 7.18 overlap | 127.6 |
| | 7 | 7.12 overlap | 125.8 |
| | NMe | 2.66 s | 28.9 |
| N—Me-His (4) | 1 | — | 171.4 |
| | 2 | 5.76 brs | 52.4 |
| | 3 | 2.84, 2.84 overlap | 40.9 |
| | 4 | — | 130.1 |
| | 5 | 7.16 overlap | 118.7 |
| | 6 | 8.15 brs | 134.4 |
| | NMe | 2.46 overlap | 28.9 |
| N—Me-Leu (5) | 1 | — | 171.0 |
| | 2 | 4.52 brs | 50.5 |
| | 3 | −0.54 m, 1.02 overlap | 34.0 |
| | 4 | 0.75 m | 23.8 |
| | 5 | 0.51 d (6.5) | 22.7 |
| | 6 | 0.39 d (6.5) | 19.0 |
| | NMe | 2.56 s | 30.0 |
| N—Me-Phe (6) | 1 | — | n.d. |
| | 2 | 4.32 brs | 61.0 |
| | 3 | 2.70, 2.91 overlap | 32.7 |
| | 4 | — | 137.9 |
| | 5 | 7.08 d (7.5) | 129.3 |
| | 6 | 8.21 t (7.5) | 128.2 |
| | 7 | 7.17 overlap | 126.2 |
| | NMe | 2.24 s | 28.0 |
| Tyr (7) | 1 | — | 170.7 |
| | 2 | 4.50 overlap | 53.0 |
| | 3 | 2.50, 2.84 overlap | 36.6 |
| | 4 | — | 127.8 |
| | 5 | 6.84 brs | 129.6 |
| | 6 | 6.55 d (8.5) | 114.6 |
| | 7 | — | 155.6 |
| | NH | 8.06 brs | — |
| 3-OH-Phe (8) | 1 | — | 170.3 |
| | 2 | 4.48 overlap | 58.5 |
| | 3 | 4.68 d (9.5) | 71.2 |
| | 4 | — | 143.2 |
| | 5 | 7.44 d (7.5) | 127.0 |
| | 6 | 7.32 t (7.5) | 127.4 |
| | 7 | 7.26 t (7.5) | 127.0 |
| | NH | 8.34 overlap | — |
| Asp (9) | 1 | — | 173.7 |
| | 2 | 5.12 brs | 45.3 |
| | 3 | 2.34, 2.46 overlap | 41.6 |
| | 4 | — | n.d. |
| | NH | 8.35 overlap | — |
| N—Me-Gly (10) | 1 | — | 168.2 |
| | 2 | 3.75 d (1.5), 4.19 overlap | 53.2 |
| | NMe | 3.27 s | 37.6 |
| Dap (11) | 1 | — | n.d. |
| | 2 | 4.52 overlap | 51.3 |
| | 3 | 2.99 m, 3.33 m | 40.2 |
| | NH | 8.82 brs | — |

The connectivity of these residues was established by analysis of HMBC and NOESY spectra. High-resolution mass spectrometry (HR-MS) and tandem HR-MS data were consistent with this structure. The former gave [M+H]⁺_{obs}=1398.6840, consistent with the molecular formula of C₇₀H₉₁N₁₅O₁₆ ([M+H]⁺_{calc}=1398.6846, Δppm=0.43).

Meanwhile tandem HR-MS revealed full sets of b-ions for three linear acylium ions obtained from fragmentation at amide bonds between N-Me-Phe3 and NMe-Phe6. Together these data established the planar structure of the cryptic metabolite.

Next, absolute configurations of stereogenic centers in each amino acid constituent were determined using Marfey's method.

Various modifications may be made to the systems, methods, apparatus, mechanisms, techniques and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof. As such, the appropriate scope of the invention is to be determined according to the claims.

What is claimed is:

1. A composition for use in treating colorectal cancer and/or adenocarcinoma, comprising:
momomycin or a derivative thereof in a pharmaceutically acceptable carrier, at a total concentration of at least 1 μM.

2. The composition according to claim 1, wherein the concentration is 1-2 μM.

3. A pharmaceutical composition comprising:
momomycin or a derivative thereof in a pharmaceutically acceptable carrier, at a total concentration of at least 1 μM.

4. The pharmaceutical composition according to claim 3, wherein the concentration is 1-2 μM.

5. A method for production of momomycin, comprising:
growing a culture of a strain of Streptomyces rimosus in the presence of phytosphingosine.

6. The method according to claim 5, wherein the strain is S. rimosus Δrim::apr^{R}.

7. The method according to claim 6, further comprising filtering supernatant from the culture.

8. The method according to claim 7, further comprising purifying the filtered supernatant.

9. A method for treating colorectal cancer and/or adenocarcinoma, comprising:
administering momomycin or a derivative thereof in a pharmaceutically acceptable carrier, at a total concentration of at least 1 μM.

10. The method according to claim 9, wherein the concentration is 1-2 μM.
